(19) United States
(12) Patent Application Publication
(10) Pub. No.: US 2006/0173425 A1
(43) Pub. Date: Aug. 3, 2006

(54) Eye drop guide

(76) Inventor: Eugene J. Meierhoefer, Salem, SC (US)

Correspondence Address: NEOCONIX, INC.;c/o INTELLEVATE, LLC, P.O. BOX 52050, MINNEAPOLIS, MN, 55402, US

(21) Appl. No.: 11/340,315

(22) Filed: Jan. 26, 2006

Related U.S. Application Data
(60) Provisional application No. 60/647,818, filed on Jan. 31, 2005.

(51) Int. Cl. A61M 35/00

(52) U.S. Cl. 604/300

(57) ABSTRACT

An eye drop guide uses a mirror to locate the orifice of a dispenser in line with the eye. The mirror is positioned at about 50 degrees from the orifice so that when a patient tilts his head and positions the mirror to see his own eye the orifice is aligned so that fluid will accurately drop into the eye. The mirror's position is determined by an arm that is attached to the dispenser. The arm may fold against the dispenser or be fixed in place. The arm may also be positioned in relationship to the dispenser by a ring or clasp attached to the arm that slips over the dispenser to secure the arm to the dispenser.

This patent application is a continuation of Provisional Patent Application Ser. No. 60/647,818, filing date Jan. 31, 2005.

### BACKGROUND

1. Field of the Invention

The field of this invention is eye drop dispensing guide.

2. Background

Dispensing medication by a person to his eye from a container often results in misses and wasted medication. Various devices have been used to align a dispenser in relation to the eye by mechanical structures such as spacers. One device is a droplet at the orifice of the dispenser used to reflect an image of the eye for alignment purposes.

### DESCRIPTION OF DRAWINGS

FIG. 1 shows an Eye Drop Guide mounted onto a dispenser bottle.

FIG. 2 shows an Eye Drop Guide with an attachment ring on a dispenser bottle.

FIG. 3A&B show a section side and top views of an Eye Drop Guide with a mirror mounted on an arm positioned at an angle to a dispensing bottle with a ring to attach it to the dispensing bottle.

FIG. 4 shows a hinged arm able to rotate into position from its rest position.

FIG. 5 shows an adjustable mirror mechanism allowing the mirror to be positioned on the hinged arm of the Eye Drop Guide.

FIG. 6 shows an adjustable mirror mechanism allowing the mirror to be positioned on the hinged arm of the Eye Drop Guide.

FIG. 7 shows an Eye Drop Guide aligned in relationship to an eye to receive dispensed fluid.

### DETAILED DESCRIPTION OF INVENTION

Optical alignment of a medication dispenser to the eye using a reflective surface allows accurate placement of the medication in the eye. One configuration is a mirrored surface attached to a dispenser at an angle to reflect an image of the eye when the dispenser is properly aligned with the eye for accurate medication dispensing. The mirror may be attached in several configurations. The mirror is shown in this description as square but may be any suitable shape. It may be removable or permanently affixed. The mirror may be at a fixed location set by an attachment structure or it may be articulated to move for adjustment and/or for storage as it is folded to the dispenser.

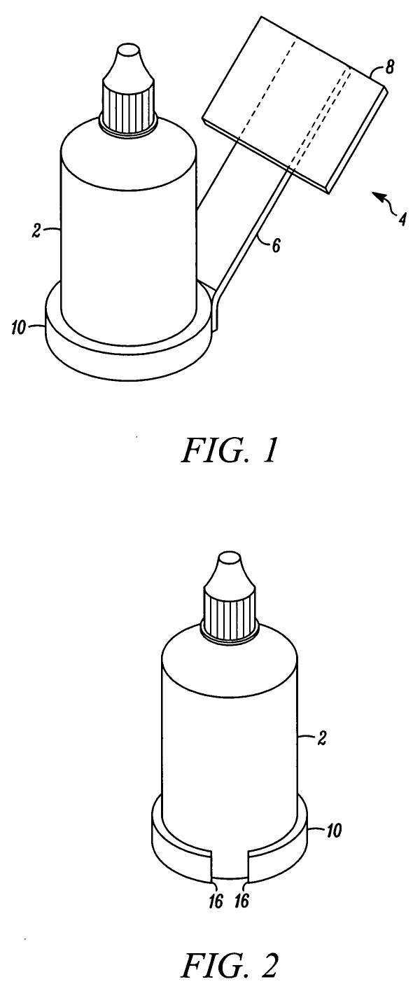

Referring to FIG. 1, a fluid dispenser, in this example a bottle, 2 for dispensing eye medication is shown with an Eye Drop Guide 4 attached to the bottle by an arm 6 that positions a mirror 8 in a spaced relationship to the bottle 2. The arm 6 is secured to the bottle 2 by an attachment ring 10 that can be compression fit onto the bottle 2. The dispenser can be other shapes such as a square, oval, etc. The shape of the dispenser 2 dictates the shape of the ring 10. For instance if the dispenser 2 is square the ring 10 would be similarly square to fit over a square dispenser 2 surface. If the dispenser 2 is oval in outer dimensions, then the ring 10 would be shaped to fit over it accordingly.

Referring to FIG. 2, ring 10 is shown on bottle 2 with a split in the ring 10. The split allows ring 10 to flex as it is slipped onto bottle 2 to secure the Eye Drop Guide 4 securely.

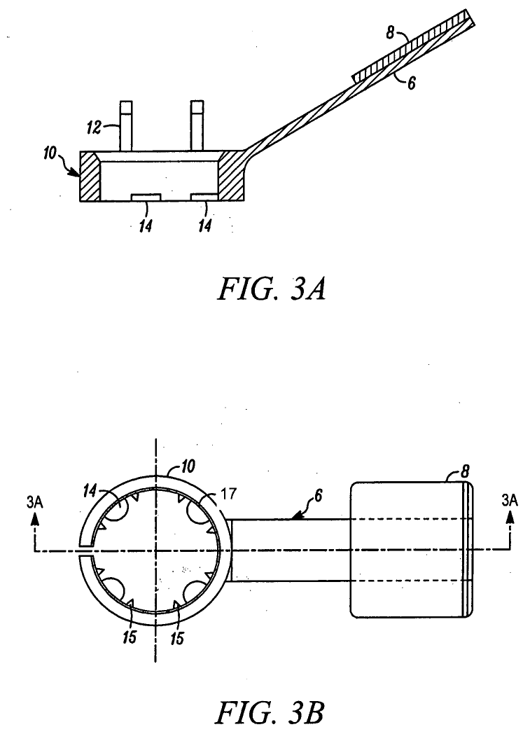

Referring to FIG. 3A, one configuration of the invention consists of a container capture ring 10 with and arm 6 extending upward from the ring 10 at an angle of approximately 50 degrees off vertical. Attached to the arm 6 is a glass mirror 8, or equivalent reflective material, e.g., highly polished metal or plastic, located at such point on the arm 6 to permit viewing of the eye when the bottle 2 is positioned at the exact location for drop administration. The Eye Drop Guide 4 may be made employing injection molded plastic, e.g. polyethylene or polypropylene, either of which may be designed to be rigid or flexible.

Another configuration is to add container guide fingers 12, projecting up from the capture ring 10, to assist in placement of the container by spreading (opening) the ring 10 as the container is inserted (mounted) into the ring 10. The fingers 12 are sized and located so as not to interfere with squeezing the container when expressing the medication.

Referring to FIG. 3B, in one configuration the dispenser 2 (not shown) is kept in proper position by a friction fit between the container 2 and the ring 10 as well as several horizontal tabs 14 extending inwardly from the bottom of the capture ring 10. Vertical depth of the ring 10 must be deep enough to firmly grip the container but short enough in height to permit squeezing a flexible container to express medication. The top edge 16 of the ring 10 is rounded or inwardly tapered slightly to facilitate placement of the dropper container into the capture ring 10. Approximately 180 degrees opposite the arm 6 the capture ring 10 contains a molded-in void 16 permitting flexing of the ring 10 to accommodate container variance as well as different container shapes. Small, thin, vertical ribs 15 molded inside the capture ring 10, help to adjust for varying container sizes, shapes, e.g., in 5 ml to 15 ml volumes.

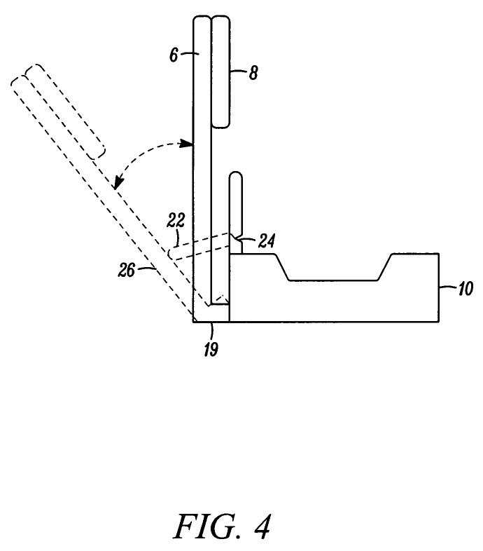

Referring to FIG. 4, a variation on the configuration of the device includes the arm 6 hinged at the capture ring 10 to permit the arm 6 to be folded in against the dispenser 2 to which it is attached. The mechanism is formed with a living hinge 19 attached to the arm 6 and arm positioning strut 22 with the arm 6 and strut 22 formed approximately parallel to one another so that there is a memory position. As the arm 6 is pulled from the nominal up position resting generally parallel to dispenser 2 the arm positioning strut 22 may bend at a hinge point 24 toward arm 6 and strut 22 may lock into strut positioning hole 26 on arm 6 to position the arm 6 and attached mirror 8 away from a dispenser 2.

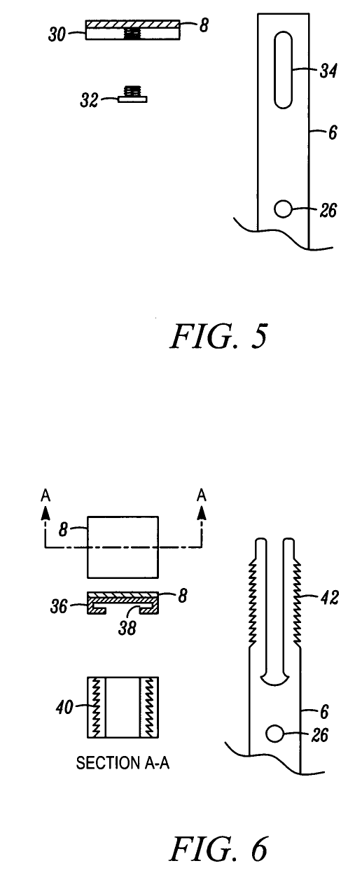

Yet another variation is a removable mirror 8, for storage and shipping. Referring to FIG. 5, mirror 8 is shown mounted on mirror plate 30 that has threads for receiving a lock-in screw 32. Mirror plate 30 is mounted on arm 6 and secured in slot 34 by lock-in screw 32 to attached mirror plate 30 to arm 6 and to adjust the position of mirror 8 by sliding mirror plate 30 along slot 34 and securing it in place by lock-in screw 32.

Referring to FIG. 6, mirror plate 36 on which mirror 8 is mounted has a slot 38 with teeth 40 along the inside walls of the slot 38. Arm 6 has serrated fingers 42 at one end to receive mirror mount 36 by inserting serrated fingers 42 into slot 38 on mirror mount 36. The serrated fingers 42 of arm 6 engage the teeth 40 in mirror mount 36 to secure mirror 8 in place. The mirror mount 36 may be moved along arm 6 to adjust the position of the mirror 8 relative to the length of arm 6.

Another variation is mounting the mirror 8 pivotally on the arm 6 so that either the mirror 8 or the arm 6, or both may pivot to allow adjustment of the mirror 8 relative to the eye or the dispenser 2.

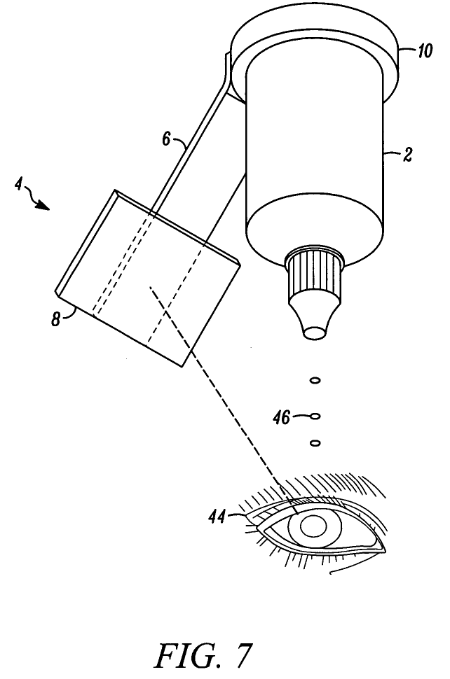

A further variation is to make the arm 6 adjustable in length by any way that allows variation in its length or position including a sliding portion of the arm 6. Referring to FIG. 7, dispenser 2 is aligned to an eye 44 by mirror 8 that is positioned so that the patient sees his eye 44 in mirror 8 when dispenser 2 is held above the eye 44. With this alignment the dispenser 2 is positioned for fluid drops 46 to be dispensed from an orifice 48 in the end of the bottle.

There are many variations of this invention in shape, material and dimensions that may be used all of which are variations of the invention described herein.

I claim:

1. An Eye Drop Guide comprising:
an adjustable ring;
an arm extending at an angle from the ring; and
a mirror attached to the arm with its reflective surface at an angle from the ring.

2. The eye drop guide of claim 1 further comprising an adjustable arm with respect to the ring.

3. The eye drop guide of claim 1 further comprising a mechanism to adjust the mirror on the arm.

4. The eye drop of claim 1 further comprising a ring having portions facing inwardly to engage a dispenser inserted in the ring.

5. A fluid dispenser comprising:
a cavity defined by a surface surrounding the cavity;
an orifice and an end to the cavity;
an arm disposed outwardly from the surface;
and a mirror attached to the arm, the mirror's reflective surface aimed towards the dispenser orifice.

6. The eye drop dispenser of claim 5 further comprising the mirror at an angle of approximately 50 degrees from a plane of the surface.

7. The eye drop guide of claim 5 further comprising an adjustable arm with respect to the ring.

8. The eye drop guide of claim 5 further comprising a mechanism to adjust the mirror on the arm.

9. An eye drop guide comprising:
a dispenser having a cavity and an outer surface;
an arm attached to the surface and angled away from the surface; and
a reflective surface on the arm facing the outer surface of the cavity.

10. The eye drop guide of claim 9 further comprising a movable arm.

11. The eye drop guide of claim 9 further comprising an arm with a pivot point at the point of attachment to the surface.

12. A method for administering eye drops to an eye from an eye drop dispenser having an arm with a mirror angled approximately 50 degrees from orifice of the dispenser comprising the steps of:
aligning the mirror to reflect the eye into which fluid from the dispenser is to placed; and
dispensing fluid into the eye.
